(19) United States
(12) Patent Application Publication
(10) Pub. No.: US 2025/0221955 A1
(43) Pub. Date: Jul. 10, 2025

(54) SMALL MOLECULES AND METHODS OF USING THE SAME FOR TREATING SKIN

(71) Applicant: L'Oreal, Paris (FR)

(72) Inventors: Amina BOUSLIMANI, Clark, NJ (US); Magali MOREAU, Aulnay-sous-Bois (FR); Mohamed Abou DONIA, Clark, NJ (US); Paola ESTRADA, Clark, NJ (US)

(21) Appl. No.: 18/982,922

(22) Filed: Dec. 16, 2024

Related U.S. Application Data
(60) Provisional application No. 63/610,954, filed on Dec. 15, 2023.

(30) Foreign Application Priority Data
Mar. 22, 2024 (FR) 2402852

(51) Int. Cl. A61K 31/352; A61K 9/00; A61K 35/74; C07D 311/80

(52) U.S. Cl. CPC A61K 31/352; A61K 9/0014; A61K 35/74; C07D 311/80

(57) ABSTRACT

The disclosure relates to new biosynthetic gene clusters that encode for type II polyketides, products thereof, extracts and compositions comprising the products, and methods of using the products, extracts, and compositions for treating skin, for example to enhance skin's barrier function and/or immune defense.

### TECHNICAL FIELD

The disclosure relates to small molecules, extracts and compositions comprising the molecules, and methods of using the same for treating skin, for example to enhance skin's barrier function and/or immune defense.

### BACKGROUND

Skin, which is the largest organ of the human body, is colonized by beneficial microorganisms and together they serve as a barrier to prevent the invasion of pathogens. In circumstances where the barrier is broken, or when the balance between commensals and pathogens is disturbed, skin diseases or disorders can result.

The human skin microbiome has approximately one thousand species of bacteria, at least some of which can disrupt or promote skin health. For example, individuals with atopic dermatitis have been shown to have an altered skin bacterial flora when compared to individuals without atopic dermatitis, with skin of individuals with atopic dermatitis dominated by increased proportions of Staphylococcus aureus as compared to those with healthy skin. In contrast, it is believed that at least some commensal bacteria have a role in protecting the skin against external aggressions and pathogens. The metabolites produced by these bacteria can in some cases inhibit pathogenic microbes and in other cases promote beneficial ones, rebalancing the microbiome of altered skin. As an example, Staphylococcus epidermidis is believed to enhance skin ceramides levels and improve skin hydration through the secretion of sphingomyelinase.

Coagulase-negative staphylococci (CoNS) are a heterogeneous group of bacteria that are less frequently associated with infections in humans than species which possess a wide range of virulence factors, such as S. aureus. For example, one strain of CoNS, Staphylococcus saccharolyticus, is part of the microbiota of the skin that rarely causes infections in humans. S. saccharolyticus requires fastidious growth conditions compared to other strains of CoNS and was only recently accurately identified. However, S. saccharolyticus has been poorly investigated to date. Another example of CONS in the skin microbiota is Staphylococcus warnei. Although considered prevalent skin colonizers, the role of S. saccharolyticus and S. warnei in skin homeostasis and skin health has been relatively uncharacterized.

Thus, there remains a need to learn more about the function of skin microbiome and its role in skin health.

In addition to the need for options to treat and/or prevent skin diseases and disorders, many consumers practice routine or preventative skin care in an effort to improve overall skin appearance or health, and/or to treat specific skin concerns. For example, consumers seek cosmetic skin care products that temporarily or permanently delay or eliminate signs of aging, counteract negative environmental influences, prevent moisture loss, etc.

Further, the design of environmentally friendly cosmetic products, which are designed and developed considering environmental issues, is becoming a major goal in an effort to meet global challenges. It is therefore essential to propose more sustainable compositions, preparation processes and ingredients to address these environmental concerns. In this context, it is important to develop new cosmetic products and processes with a better carbon footprint, particularly by promoting the use of renewable raw materials and/or cosmetic compositions with a good index of naturalness and/or materials of natural origin and, more particularly, reducing the use of compounds of petrochemical origin.

Therefore, there is an ongoing need for improved skin treatments and products. It has now been surprisingly and unexpectedly discovered that compositions comprising a purified fraction of bacteria enriched with a new series of polycyclic aromatic polyketides can improve skin health and appearance.

### SUMMARY

The disclosure relates to new biosynthetic gene clusters that encode for type II polyketides, as well as products thereof, to extracts and compositions comprising the products, and to methods for treating skin with the products, for example to improve or enhance the skin's barrier function, improve the skin's immune defense, and/or treat various types of skin diseases or skin conditions.

The disclosure also relates to a series of novel polycyclic aromatic polyketides (“Compound Series 1”), to extracts and compositions comprising the molecules, and to methods for treating skin with the molecules, for example to improve or enhance the skin's barrier function, improve the skin's immune defense, and/or treat various types of skin diseases or skin conditions.

The disclosure further relates to S. saccharolyticus bacterial extracts, for example skin-derived bacterial extracts, to compositions comprising the bacterial extracts, and to methods for treating skin with the bacterial extracts and/or compositions, for example to improve or enhance the skin's barrier function, improve the skin's immune defense, and/or treat various types of skin diseases or skin conditions.

The disclosure also relates to S. warnei bacterial extracts, for example skin-derived bacterial extracts, to compositions comprising the bacterial extracts, and to methods for treating skin with the bacterial extracts and/or compositions, for example to improve or enhance the skin's barrier function, improve the skin's immune defense, and/or treat various types of skin diseases or skin conditions.

In some embodiments, the methods of treating skin are methods for enhancing skin barrier function. In further embodiments, the methods of treating skin are methods for enhancing skin immune defense. In still further embodiments, the methods of treating skin are methods for treating a skin disease, condition, or disorder. For example, the methods may be methods of treating atopic dermatitis.

Products of the novel biosynthetic gene clusters that encode for type II polyketides, including Compound Series 1 molecules, S. saccharolyticus bacterial extracts, and S. warnei bacterial extracts can also be used as a biomarker. Thus, the disclosure further relates to methods of detecting skin diseases, disorders, or conditions, for example atopic dermatitis.

### BRIEF DESCRIPTION OF THE FIGURES

FIG. 1 shows the genetic organization of TII-PKS BGC5 in S. saccharolyticus and S. warnei.

FIG. 2 shows UV absorbance of fractions of purified S. saccharolyticus. HPLC peaks that absorb at 400 nm can only be seen in the cell extracts of TSB-grown cultures.

FIGS. 3A-3C show UV (3A), NMR (3B), and mass spectroscopy (3C) characterizing data for a novel Compound Series 1 molecule.

FIGS. 4A-4B show graded results of up-regulation of genes associated with skin barrier function (FLG, IVL, and LOR), and genes associated with cell differentiation (CLDN1, TGM1, KRT16, and KRT6A) in primary keratinocytes from healthy donors and/or the same keratinocytes treated with S. aureus.

FIGS. 5A-5B show graded results of increase the expression of antimicrobial peptides and innate immune genes, involved in immune defense (S100A7, RNASE7, DEFB4A, and PI3) in primary keratinocytes from healthy donors and/or the same keratinocytes treated with S. aureus.

### DETAILED DESCRIPTION

The disclosure relates to new biosynthetic gene clusters that encode for type II polyketides, products thereof including a series of novel molecules, bacterial extracts containing the products and/or molecules, compositions comprising the products and/or molecules, compositions comprising the bacterial extracts, and methods of treating the skin. The products and/or molecules can also be used as biomarkers to detect skin diseases.

### I. Molecules, Extracts, and Compositions

In various embodiments, the disclosure relates to bacterial extracts. In particular, the disclosure relates to S. saccharolyticus and S. warnei bacterial extracts, for example skin-derived bacterial extracts. The extracts are preferably purified. In further embodiments, the disclosure relates to products in the extracts, for example products of biosynthetic gene clusters (“BCGs”) that encode for type II polyketides (“TII-PK”).

Polyketides are secondary metabolites that have diverse chemical structures and a broad variety of biological activities, for example antibacterial, antifungal, anticancer, antiviral, immune-suppressing, anti-cholesterol, and anti-inflammatory activity. Polyketides can be obtained from a variety of sources, including bacteria.

Biosynthesis of polyketides is very complex because the process involves multifunctional enzymes called polyketide synthases (“PKSs”). The genes responsible for polyketide biosynthesis are clustered in the genome, forming BGCs. Each BGC encodes the PKS responsible for the formation of the carbon backbone, together with the tailoring enzymes required for primary tailoring events, subsequent tailoring events to form the final polyketide structure, as well as genes encoding the regulation of the BGC and resistance to the end product if applicable.

Bacterial aromatic polyketides are generally synthesized by type II PKSs (“TII-PKS”), which are not as well understood as type I or type III PKSs. However, skin-derived TII-PKs are not known to have effects for topical skin applications.

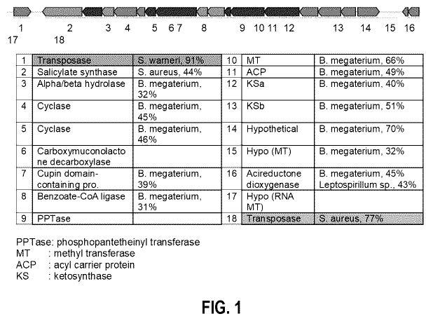

A novel TII-PKS BGC, TII-PKS BGC5 (FIG. 1) has now been discovered. It is believed that the products of this organism have properties which enhance skin's barrier function and/or immune defense. These properties may be beneficial for treating certain skin conditions such as atopic dermatitis. One such product, a novel small molecule (“Compound Series 1”), which has been purified from an active fraction of skin-derived S. saccharolyticus bacterial extracts, has the following structure:

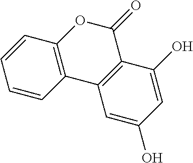 [SMILES: O=c1oc2ccccc2c2cc(O)cc(O)c12]

Thus, in various embodiments the disclosure relates to products of TII-PKS BGC5, including Compound Series 1 molecules, and extracts and/or compositions comprising the products.

It has surprisingly been discovered that TII-PKS BGC5 is not present on the skin of individuals with atopic dermatitis, but is present in healthy individuals. Thus, it is believed that products of TII-PKS BGC5, including Compound Series 1 molecules, may have beneficial effects for treating atopic dermatitis.

It has also been surprisingly discovered that, under basal and stimulated conditions mimicking atopic dermatitis in vitro, these purified S. saccharolyticus extracts induce, in keratinocytes, an up-regulation of genes associated with skin barrier function (Filaggrin, Involucrin, and Loricrin), cell differentiation (Claudin 1, Corneodesmosin, Keratin 16, and Keratin 6A type I), with antimicrobial peptide production, and with innate immune response (S100A7, RNASE7, DEFB4A, and PI3). These discoveries suggest that skin barrier function and/or immune defense can be enhanced by treatment with the S. saccharolyticus and S. warnei extracts, products of TII-PKS BGC5, and/or Compound Series 1 molecules in vivo.

Thus, in various embodiments, the disclosure further relates to bacterial extracts containing TII-PKS BGC5 and/or products thereof, including Compound Series 1 molecules, compositions comprising TII-PKS BGC5 and/or products thereof, in particular obtained from S. saccharolyticus or S. warnei, for example skin-derived S. saccharolyticus and S. warnei, and compositions comprising the extracts.

The compositions may, in various embodiments, be topical compositions. The form of the compositions can vary. For example, the composition may be a cream, a lotion, a gel, a hydrogel, an ointment, a paste, a serum, a foam, a spray, a film, or any suitable form for topically administering TII-PKS BGC5 and/or a product thereof, including Compound Series 1 molecules, or an extract described herein to the skin. The compositions may comprise one or more additional components suitable for topically administering TII-PKS BGC5 and/or a product thereof, including Compound Series 1 molecules, or an extract described herein to the skin, or suitable for treating skin, for example one or more cosmetically or pharmaceutically acceptable carriers or vehicles (e.g. water and/or non-aqueous solvents such as glycols), penetration or absorption enhancers, solubility enhancers, pH adjusters, emollients, antioxidants, vitamins, preservatives, etc.

The compositions may, in various embodiments, comprise TII-PKS BGC5 and/or a product thereof in an amount ranging from about 0.0001%, such as from about 0.0005%, from about 0.001%, or from about 0.005%, up to about 25%, such as up to about 18%, up to about 15%, up to about 12%, up to about 10%, up to about 8%, up to about 5%, up to about 4%, up to about 3%, up to about 2.5%, up to about 2%, up to about 1.9%, up to about 1.8%, up to about 1.7%, up to about 1.6%, up to about 1.5%, up to about 1.4%, up to about 1.3%, up to about 1.2%, up to about 1.1%, up to about 1%, up to about 0.9%, up to about 0.8%, up to about 0.7%, up to about 0.6%, up to about 0.5%, up to about 0.4%, up to about 0.3%, up to about 0.2%, up to about 0.1%, up to about 0.09%, up to about 0.08%, up to about 0.07%, up to about 0.06%, up to about 0.05%, up to about 0.04%, up to about 0.03%, up to about 0.02%, or up to about 0.01% by weight, based on the total weight of the composition, including all ranges and subranges using any of the foregoing as upper and lower limits.

The extracts may, in various embodiments, comprise TII-PKS BGC5 and/or a product thereof in an amount ranging from about 1%, such as from about 5%, from about 10%, from about 15%, from about 20%, or from about 25%, up to about 99.99%, such as up to about 99.95%, up to about 99.9%, up to about 99.5%, up to about 99%, up to about 98%, up to about 97%, up to about 96%, up to about 95%, up to about 94%, up to about 93%, up to about 92%, up to about 91%, up to about 90%, up to about 85%, up to about 80%, up to about 75%, up to about 70%, up to about 65%, up to about 60%, up to about 55%, up to about 50%, up to about 45%, up to about 40%, up to about 35%, or up to about 30% by weight, based on the total weight of the extract, including all ranges and subranges using any of the foregoing as upper and lower limits.

### II. Methods of Treating Skin

The disclosure also relates to methods of treating skin. As described herein, TII-PKS BGC5 and/or products thereof, including Compound Series 1 molecules, and extracts and/or compositions containing the same, particularly those obtained from skin-derived S. saccharolyticus or S. warnei, are believed to enhance skin barrier function and/or immune defense, and/or may be useful for treating certain skin conditions such as atopic dermatitis. Various methods of treating skin according to the disclosure therefore relate to: (i) methods of enhancing skin barrier function, (ii) methods of enhancing the immune defense of skin, (iii) methods of treating skin diseases, disorders, and/or conditions, and/or (iv) methods of treating atopic dermatitis.

In some embodiments, the foregoing methods comprise applying TII-PKS BGC5 and/or a product thereof, for example a Compound Series 1 molecule, or an extract or composition containing the same, particularly those obtained from skin-derived S. saccharolyticus or S. warnei, to skin.

In other embodiments, the foregoing methods comprise applying TII-PKS BGC5 and/or a product thereof, for example a Compound Series 1 molecule, or an extract or composition containing the same, particularly those obtained from skin-derived S. saccharolyticus or S. warnei, to the skin of an individual in need of one or more of: (i) enhancement of skin barrier function, (ii) enhancement of the immune defense of skin, (iii) treatment of a skin disease, disorder, and/or condition, and/or (iv) treatment of atopic dermatitis.

As described herein, treatment of skin diseases, disorders, and/or conditions, such as atopic dermatitis, is intended to mean treating the disease, disorder, and/or condition, such as atopic dermatitis, itself, and/or treating one or more symptoms or associated physical effects of disease, disorder, and/or condition. By way of example, symptoms or associated physical effects of atopic dermatitis may include pruritus, dryness, inflammation, etc. As such, a method of treating atopic dermatitis may include treating the skin in such a manner as to alter the skin's microbiome to decrease or minimize the incidence of atopic dermatitis, or may include treating the skin in such a manner as to decrease, minimize, and/or eliminate one or more of such symptoms, e.g. pruritus, dryness, inflammation, etc.

The methods may comprise applying TII-PKS BGC5 and/or a product thereof, for example a Compound Series 1 molecule, or an extract or composition containing the same, one or more times per day, for example two or more times per day, three or more times per day, etc., as needed or desired. The time period for which such application may continue is not limited. The application may occur one or more times per day, for example at least twice per day or at least three times per day, for a period of at least 2 days, such as at least 3 days, at least 5 days, at least 7 days, at least 10 days, at least 12 days, at least two weeks, at least three weeks, at least four weeks, at least five weeks, at least 6 weeks, at least two months, at least three months at least four months, at least five months, at least six months, at least nine months, at least 12 months, at least 18 months, at least two years, etc., as needed to provide the desired effect, e.g. skin barrier enhancement, skin immune defense enhancement, treatment of atopic dermatitis, etc.

It will be apparent that modifications and variations of the embodiments herein are possible without departing from the scope of the disclosure. Furthermore, it should be appreciated that all examples in the present disclosure, while illustrating various embodiments of the disclosure, are provided as non-limiting examples and are, therefore, not to be taken as limiting the various aspects so illustrated. It is to be understood that all definitions herein are provided for the present disclosure only.

As used herein, the terms “treating,” “treatment,” or the like, with regard to skin diseases, disorders, and conditions are intended to include any degree of treatment.

As used herein, the terms “enhance,” “enhancement,” or the like, with regard to skin barrier function, are intended to mean any improvement or increase of the treated skin's epidermal differentiation, pathogen barrier function, and/or water barrier function.

As used herein, the terms “enhance,” “enhancement,” or the like, with regard to skin's immune defense system, are intended to mean any improvement or increase of the treated skin's ability to maintain a healthy balance of the microbiome and/or reduce, inhibit, or eliminate pathogenic microbes.

As used herein, the term “extract” is intended to mean a formulation or blend of compounds isolated from S. saccharolyticus or S. warnei.

As used herein, the term “purified extract” is intended to mean a concentrated formulation or blend of compounds isolated from S. saccharolyticus or S. warnei.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” means “at least one” and thus includes individual components as well as mixtures/combinations. Thus, where the disclosure refers to “an element selected from the group consisting of A, B, C, D, E, F, a salt thereof, or mixtures thereof,” it indicates that one or more of A, B, C, D, and F may be included, one or more of a salt of A, a salt of B, a salt of C, a salt of D, a salt of E, or a salt of F may be included, or a mixture of any two of A, B, C, D, E, F, a salt of A, a salt of B, a salt of C, a salt of D, a salt of E, and a salt of F may be included.

The term “and/or” should be understood to include both the conjunctive and the disjunctive. For example, “treat and/or prevent” means “treat and prevent” as well as “treat or prevent,” and expressly covers instances of either.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value, unless otherwise stated.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Unless expressly stated otherwise, “about” means +/−5%. Likewise, all endpoints of ranges are understood to be individually disclosed, such that, for example, a range of 1:2 to 2:1 is understood to disclose a ratio of both 1:2 and 2:1.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it not intended that any particular order be inferred.

The examples that follow serve to illustrate embodiments of the present disclosure without, however, being limiting in nature. It will be apparent to those skilled in the art that various modifications and variations can be made in the compositions and methods of the invention without departing from the spirit or scope of the invention. Thus, it is intended that the present disclosure covers the modifications and variations that come within the scope of the appended claims and their equivalents.

### EXAMPLES

The following Examples are intended to be non-limiting and explanatory in nature only.

### Example 1—Identification of a Novel TII-PK BCG Producer Organism

Using a known algorithm (MetaBGC, (9^{§§})) and metagenomic sequencing data (28), samples taken from skin of individuals with (AD-positive) and without (AD-negative) atopic dermatitis were studied. The results of these studies are shown in Table 1:

TABLE 1

| | TII-PKS | TII-PKS | |
|---|---|---|---|
| | plasmid- | plasmid- | |
| | positive | negative | TOTAL |
| AD-positive (n = 350) | 0 | 350 | 350 |
| AD-negative (n = 1126) | 36 | 1090 | 1126 |
| Total (n = 1476) | 36 | 1440 | 1476 |
| Risk* | 0** | 0.243** | 0.237 |

*Risk ratio, 0
**p = 4e⁻⁵ by one-sided Fisher's exact test

This in silico analysis shows that TII-PKS-BGC5 is only present in individuals with healthy (AD-negative) skin. Genetic organization for TII-PKS-BGC5 is shown in FIG. 1 (showing annotation of each gene as well as the closed NCBI hit).

### Example 2—Identification of Compound Series 1 Molecule

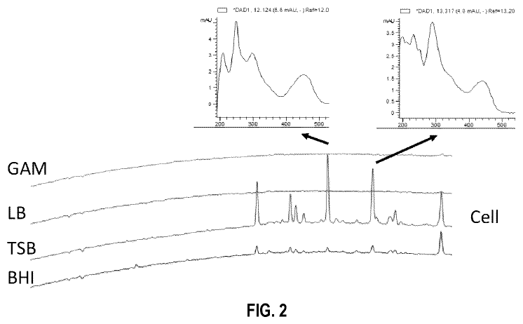

About 100 L of a strain of S. saccharolyticus with confirmed presence of TII-PKS-BGC5 was cultured in Tryptic Soy Broth (TSB) under anaerobic conditions at 37° C. After five days, cell pellets were extracted by repetitive sonication in ethyl acetate, then drying the extract under vacuum. The dried crude extract was then fractionated on several chromatography columns to obtain fractions of varying polarity, and the fractions were evaluated with UV absorbance (FIG. 2).

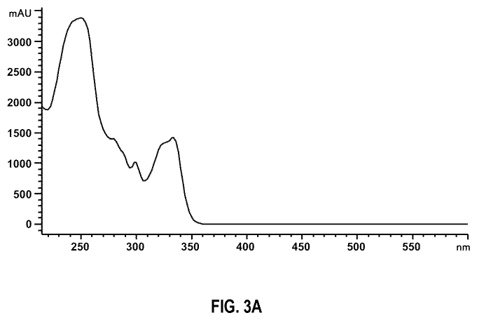

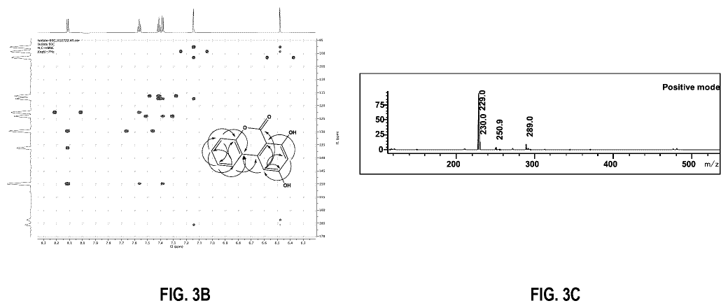

A novel Compound Series 1 molecule was found in one of the fractions, identified by UV absorbance (FIG. 3A), HMBC (Heteronuclear Multiple Bond Correlation) NMR spectroscopy (FIG. 3B), and mass spectroscopy (FIG. 3C).

### Example 3—Biological Activity of Compound Series 1 Molecule

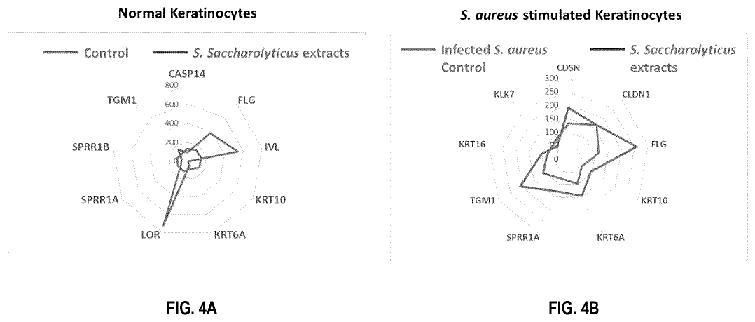

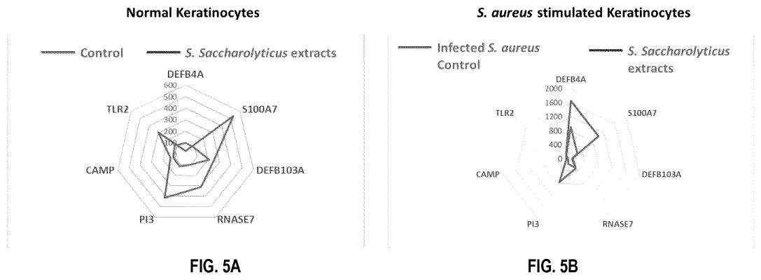

In order to assess biological activity of the Compound Series 1 molecule identified in Example 2, in vitro models of 2D primary human epidermal keratinocytes from a healthy donor (“normal model”) and the same keratinocytes stimulated with S. aureus (“AD-like model”) were used. Both basal and infected NHEK were treated with S. saccharolyticus extract (n=3). RNA was extracted from the cell cultures treated with S. saccharolyticus extract and untreated (control) 48 hours after incubation and qPCR analysis was performed for gene expression analysis. The results of these studies are shown in FIGS. 4A-4B and 5A-5B (y-axis represents relative gene expression).

FIGS. 4A-4B show that the purified S. saccharolyticus extracts induce an up-regulation of genes associated with skin barrier function (FLG, IVL, and LOR), and genes associated with cell differentiation (CLDN1, TGM1, KRT16, and KRT6A) in normal and/or AD-like models. FIGS. 5A-5B show that the purified S. saccharolyticus extracts increase the expression of antimicrobial peptides and innate immune genes, involved in immune defense (S100A7, RNASE7, DEFB4A, and PI3) in normal and/or AD-like models.

The above results demonstrate that a skin-derived S. saccharolyticus bacterial extract surprisingly enhances skin barrier function and immune defense in atopic dermatitis models in vitro. Based on these results, it is believed that Compound Series 1 molecules, an S. saccharolyticus bacterial extract, particularly skin-derived, from strains encoding Compound Series 1, and compositions comprising Compound Series 1 molecules and/or the extract can be used in vivo to enhance skin barrier function and immune defense, and treat skin diseases, disorders, and conditions such as atopic dermatitis.

The following are incorporated by reference herein:
(1) Ahle C M, Stødkilde K, Afshar M, Poehlein A, Ogilvie L A, Söderquist B, Hüpeden J, Brüggemann H. Staphylococcus saccharolyticus: An Overlooked Human Skin Colonizer. Microorganisms. 2020 Jul. 23; 8 (8): 1105 (doi: 10.3390/microorganisms8081105);
(2^{Ø}) Byrd A L, Belkaid Y, Segre J A. The human skin microbiome. Nat Rev Microbiol. 2018 March; 16 (3): 143-155 (doi: 10.1038/nrmicro.2017.157);
(3) Nakatsuji T, Chen T H, Narala S, Chun K A, Two A M, Yun T, Shafiq F, Kotol P F, Bouslimani A, Melnik A V, Latif H, Kim J N, Lockhart A, Artis K, David G, Taylor P, Streib J, Dorrestein P C, Grier A, Gill S R, Zengler K, Hata T R, Leung D Y, Gallo R L. Antimicrobials from human skin commensal bacteria protect against Staphylococcus aureus and are deficient in atopic dermatitis. Sci Transl Med. 2017 Feb. 22; 9 (378): eaah4680 (doi: 10.1126/scitranslmed.aah4680);
(4) Nakatsuji T, Cheng J Y, Gallo R L. Mechanisms for control of skin immune function by the microbiome. Curr Opin Immunol. 2021 October; 72:324-330 (doi: 10.1016/j.coi.2021.09.001);
(5) Nakatsuji T, Gallo R L. Ann Allergy Asthma Immunol. The role of the skin microbiome in atopic dermatitis. 2019 March; 122 (3): 263-269 (doi: 10.1016/j.anai.2018.12.003);
(6) Nodake Y, Matsumoto S, Miura R, Honda H, Ishibashi G, Matsumoto S, Dekio I, Sakakibara R. Pilot study on novel skin care method by augmentation with Staphylococcus epidermidis, an autologous skin microbe—A blinded randomized clinical trial. J Dermatol Sci. 2015 August; 79 (2): 119-26 (doi: 10.1016/j.jdermsci.2015.05.001);
(7) O'Neill A M, Nakatsuji T, Hayachi A, Williams M R, Mills R H, Gonzalez D J, Gallo R L. Identification of a Human Skin Commensal Bacterium that Selectively Kills Cutibacterium acnes. J Invest Dermatol. 2020 August; 140 (8): 1619-1628.e2 (doi: 10.1016/j.jid.2019.12.026);
(8) Simonart T, Dramaix M, De Maertelaer V. Efficacy of tetracyclines in the treatment of acne vulgaris: a review. Br J Dermatol. 2008 February; 158 (2): 208-1 (doi: 10.1111/j.1365-2133.2007.08286);
(9^{§§}) Sugimoto Y, Camacho F R, Wang S, Chankhamjon P, Odabas A, Biswas A, Jeffrey P D, Donia M S. A metagenomic strategy for harnessing the chemical repertoire of the human microbiome. Science. 2019 Dec. 13; 366 (6471): eaax9176 (doi: 10.1126/science.aax9176);
(10) Zheng Y, Hunt R L, Villaruz A E, Fisher E L, Liu R, Liu Q, Cheung G Y C, Li M, Otto M. Commensal Staphylococcus epidermidis contributes to skin barrier homeostasis by generating protective ceramides. Cell Host Microbe. 2022 Mar. 9; 30 (3): 301-313.e9 (doi: 10.1016/j.chom.2022.01.004);
(11) Zhu X, Siitonen V, Melançon lii CE, Metsä-Ketelä M. ACS Synth Biol. Biosynthesis of Diverse Type II Polyketide Core Structures in Streptomyces coelicolor M1152. 2021 Feb. 19; 10 (2): 243-251 (doi: 10.1021/acssynbio.0c00482).

1-56. (canceled)

57. A biosynthetic gene cluster (BGC) that encodes for type II polyketides (TII-PK) having the genetic organization as shown in FIG. 1 (TII-PKS BGC5).

58. A compound encoded by TII-PKS BGC5 of claim 57

59. A compound according to claim 58, having the formula:

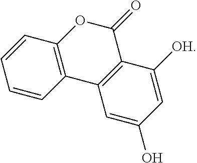 [SMILES: O=c1oc2ccccc2c2cc(O)cc(O)c12]

60. An S. saccharolyticus or S. warnei bacterial extract comprising TII-PKS BGC5 of claim 57.

61. The S. saccharolyticus or S. warnei bacterial extract of claim 60 comprising a compound of claim 59.

62. The S. saccharolyticus or S. warnei bacterial extract of claim 61, comprising a total amount of said compound(s) in an amount ranging from about 1% to about 99% by weight, based on the total weight of the extract.

63. The S. saccharolyticus or S. warnei bacterial extract of claim 62, comprising a total amount of said compound(s) in an amount ranging from about 5% to about 99% by weight, based on the total weight of the extract.

64. The S. saccharolyticus or S. warnei bacterial extract of claim 59, comprising a total amount of said compound(s) in an amount ranging from about 15% to about 99.99%, preferably to about 99.95%, more preferably to about 99.9%, more preferably still to about 99.5%, most preferably to about 99% by weight, based on the total weight of the extract.

65. The S. saccharolyticus or S. warnei bacterial extract of claim 59, comprising a total amount of said compound(s) in an amount ranging from about 25% to about 99.99%, preferably to about 99.95%, more preferably to about 99.9%, more preferably still to about 99.5%, most preferably to about 99% by weight, based on the total weight of the extract.

66. A composition comprising a compound of claim 59.

67. The composition of claim 66, comprising a total amount of said compound(s) in an amount ranging from about 0.0001% to about 2% by weight, based on the total weight of the composition.

68. The composition of claim 66, comprising a total amount of said compound(s) in an amount ranging from about 0.001% to about 2% by weight, based on the total weight of the composition.

69. The composition of claim 66, comprising a total amount of said compound(s) in an amount ranging from about 0.0001% to about 1.5% by weight, based on the total weight of the composition.

70. The composition of claim 66, comprising a total amount of said compound(s) in an amount ranging from about 0.0005% to about 0.1% by weight, based on the total weight of the composition.

71. The composition of claim 66, which is a topical composition.

72. The composition of claim 71, further comprising at least one additional component chosen from cosmetically or pharmaceutically acceptable carriers or vehicles, penetration enhancers, absorption enhancers, solubility enhancers, pH adjusters, emollients, antioxidants, vitamins, preservatives, or combinations of two or more thereof.

73. A composition comprising the S. saccharolyticus or S. warnei bacterial extract of claim 60.

74. A method of treating a skin disease, skin condition or skin disorder in a subject, comprising applying a compound of claim 59 to the skin of the subject.

75. A method of treating atopic dermatitis, the method comprising:
(a) applying a compound encoded by the TII-PKS BGC5; or
(b) applying a bacterial extract comprising said TII-PKS BGC5; or
(c) applying a composition comprising said compound or bacterial extract.

76. A method of identifying a disease, disorder, or condition of the skin, the method comprising:
(a) using a compound encoded by the TII-PKS BGC5; or
(b) using a bacterial extract comprising said TII-PKS BGC5; or
(c) using a composition comprising said compound or bacterial extract.
